(19) United States
(12) Patent Application Publication
(10) Pub. No.: US 2003/0220557 A1
(43) Pub. Date: Nov. 27, 2003

(54) Image guided liver interventions based on magnetic tracking of internal organ motion

(76) Inventors: Kevin Cleary, Washington, DC (US); Filip Banovac, Washington, DC (US); Brad Wood, Bethesda, MD (US); Elliot Levy, Washington, DC (US)

Correspondence Address: Merchant & Gould P.C., P.O. Box 2903, Minneapolis, MN, 55402-0903, US

(21) Appl. No.: 10/377,528

(22) Filed: Feb. 28, 2003

Related U.S. Application Data
(60) Provisional application No. 60/360,983, filed on Mar. 1, 2002.

(51) Int. Cl. A61B 5/05

(52) U.S. Cl. 600/409; 600/425

(57) ABSTRACT

Described is a method of providing image guidance for use in an organ or area of interest subjected to motion that includes acquiring a three-dimensional image of the organ or area of interest of the subject with at least two imageable, visible markers and at least one magnetically tracked marker in place, acquiring a three-dimensional image of the organ or area of the subject with at least two imageable, visible markers and at least one magnetically tracked marker in place, correlating a magnetic field space to the three-dimensional image space, providing an overlay of a magnetically tracked probe in the three-dimensional image space, planning a path to a target within the organ or area of interest within the subject, and proceeding along the planned path.

This application claims priority to U.S. Provisional Application Serial No. 60/360,983, filed Mar. 1, 2002 entitled Image Guided Liver Interventions Based on Magnetic Tracking of Internal Organ Motion, the disclosure of which is incorporated herein by reference.

This invention may be supported by the Department of Health and Human Services. The Government of the United States of America may have certain rights in the invention disclosed and claimed herein below.

### FIELD OF THE INVENTION

The invention relates generally to invasive medical procedures using interventional radiology. More specifically, the invention relates to medical procedures for image-guided abdominal intervention using magnetic tracking of internal organ motion and graphical depiction of surgical instruments.

### BACKGROUND OF THE INVENTION

Minimally invasive abdominal interventions are rapidly increasing in popularity. This is due to the development of new interventional techniques and the desire on the part of both clinicians and patients to decrease procedure related morbidity and trauma. Minimally invasive interventions are done using catheters, needles, or other instruments that are introduced, targeted, and manipulated without the benefit of the direct instrument visualization afforded by the usual surgical exposure. This greatly minimizes trauma to the patient, but severely restricts the physician's view of the underlying anatomy. Image-guided surgery, however, circumvents this encumbrance. It uses preoperative magnetic resonance imaging (MRI) or computed tomography (CT) scans to guide invasive surgical procedures.

Over the past decade, minimally invasive hepatic interventions have played an increasingly important role in the care of patients with primary or metastatic hepatic malignancies and complications of hepatic cirrhosis. Transhepatic biliary drainage, intrahepatic portosystemic shunt creation, and hepatic chemoembolization are being performed with increasing frequency for biliary duct obstruction, portal hypertension, and hepatic neoplasms respectively. In many cases biliary duct or portal vein puncture is successful only after multiple needle punctures using conventional fluoroscopy. An image-guided catheter or instrument placement system could play an important role in future intrahepatic or vascular interventions, both in improving the ease and accuracy of existing interventions and in enabling new interventions. Implementing an image-guided system with magnetic tracking of organ motion could also permit respiratory-gated needle placement.

The current state of the art in image guided surgery systems is based on bony landmarks with applications in the brain and spine. One example of a device used for guiding invasive surgical procedures is seen in U.S. Pat. No. 5,558,091. The system described therein includes a magnetic positioning system that utilizes a reference probe, an instrument probe, and a magnetic field to magnetically track the instrument probe in the area of interest. This system does not offer the user a method that includes the option of planning a path to the target and computer guided assistance for reaching the target.

As such there is a need for an image guidance system for use in an organ or an area of interest which provides path planning capabilities and real-time tracking of the user's probe or instrument.

### SUMMARY OF THE INVENTION

The invention provides a method of providing image guidance, for use in an organ or area of interest subjected to motion that includes acquiring a three-dimensional image of the organ or area of interest of the subject with at least two imageable, visible markers and at least one magnetically tracked marker in place, acquiring a three-dimensional image of the organ or area of the subject with at least two imageable, visible markers and at least one magnetically tracked marker in place, correlating a magnetic field space to the three-dimensional image space, providing an overlay of a magnetically tracked probe in the three-dimensional image space, planning a path to a target within the organ or area of interest within the subject, and proceeding along the planned path.

One embodiment of the invention includes a method where proceeding along the planned path includes use of a graphical user interface.

### BRIEF DESCRIPTION OF THE DRAWINGS

FIGS. 1a, b, and c display the AURORA® control unit and field generator (FIG. 1a), sensors compared to a match (FIG. 1b), and measurement volume (FIG. 1c). (all figures courtesy of Northern Digital)

FIG. 2 displays one embodiment of a graphical user interface for use in a method of the invention.

FIG. 3 displays an image of a liver reparatory motion simulator.

FIG. 4 displays the MagTrax needle/probe combination with a stylette containing a magnetic sensor in its tip and leads existing in the hub, with an 18-gauge trocar shown on the right for comparison.

FIGS. 5a, b, c and d display points in the step of planning and executing a path to the target.

FIGS. 6a and b display fluoroscopy images showing the needle puncture. FIG. 6a is an anterior-posterior view. The needle enters from the left and outline of straws can faintly be seen in middle. FIG. 6b is a lateral view. The needle enters from the left and passes through the two straws which form an X. The catheter can also be seen in this figure.

FIG. 7 displays orthogonal biplane fluoroscopic images of the liver phantom, which confirmed successful puncture of both targets by the single needle pass.

FIG. 8 displays orthogonal biplane digital images obtained for each needle pass to confirm successful target puncture.

FIG. 9 displays images of a 0.035 inch guidewire through the needle into the targeted “vessel”.

FIG. 10 displays a picture of the interventional suite and experimental set-up.

### DETAILED DESCRIPTION OF THE PREFERRED EMBODIMENT

The invention includes methods of monitoring and directing the position of a probe in a subject. One embodiment of the invention includes the steps of acquiring a three-dimensional image of the organ or area of the subject with imageable, visible markers in place, correlating a magnetic field space to the three-dimensional image space, providing an overlay of a magnetic probe in the three-dimensional image space, planning a path to the target, and proceeding along the planned path.

Acquiring a Three-Dimensional Image

The step of obtaining a three-dimensional image of the organ or area of interest functions to provide a three-dimensional image of the organ or area of interest that may provide a frame of reference for the magnetic field generated space. In an instance where the method is being carried out to, for example, target a tumor, the three-dimensional image provides a way of locating the tumor within the organ or area of interest even with motion of the organ or area of interest.

One step of a method of the invention includes obtaining a three-dimensional image of the organ or area of the subject. Virtually any method of obtaining a three-dimensional image that is commonly used can be utilized in the method of the invention. Examples of such methods include CT imaging, rotational angiography and the like. In an embodiment of the invention where the three-dimensional image is a CT image, the CT image can be obtained by any protocol that is commonly used.

The three-dimensional image is acquired with at least two imageable, visible markers and at least one magnetically tracked marker in place within the area of the organ or area of interest that is to be imaged. In one embodiment, three imageable, visible markers are utilized along with one magnetically tracked marker. In another embodiment, two of the imageable, visible markers are on the surface of the organ or area of interest to be imaged, and one is at some depth below the surface of the organ or area of interest to be imaged. The imageable markers and at least one magnetically tracked marker are maintained in the same position relative to the organ or area of interest to be imaged. In other words, the imageable, visible markers and the at least one magnetically tracked marker move with respiration but do not move with respect to the organ or area of interest that they are attached to or imbedded in.

Any markers that are imageable with the particular three-dimensional image that is being acquired, and visibly apparent can be utilized as the imageable markers. As used herein, a marker that is imageable is one that can be recognized on a computer generated depiction of the three-dimensional image that was obtained. As used herein, a marker that is visibly apparent is one that can be visually detected by the user. In one embodiment of the invention, one example of a marker that is imageable by, for example CT imaging, and visibly apparent includes skin fiducials or multimodality markers from IZI Medical, Baltimore Md.

The at least one magnetically tracked marker functions to monitor the location of the organ or area of interest as the method is carried out. Because the magnetically tracked marker is stationary within the organ or area of interest, magnetic tracking of it provides magnetic tracking of the organ or area of interest. As used herein, a magnetically tracked marker is one whose location can be monitored by the magnetic field generator tracking system that is used in the method. Examples of such magnetically tracked markers include sensors that are a part of the AURORA™ device. Specifically, these sensors can be cylindrically shaped sensors with dimensions of about 0.9 mm by 8 mm. Examples of such sensors and systems can be found at least in U.S. Pat. No. 6,288,785 (Frantz et al.) which is incorporated herein by reference.

Both the imageable, visually apparent markers and the magnetically tracked marker are placed on the organ or area of interest before the three-dimensional image is acquired, and are not moved until the method has been completed, or the desired procedure has been completed.

Correlating the Magnetic Field Space with the Three-Dimensional Image Space

The next step in a method of the invention includes correlating the magnetic field space with the three-dimensional image space, which functions to provide overlapping positions within the three-dimensional imaged space and the magnetic field space.

In one embodiment, this can be accomplished by use of a device that includes a magnetic field generator and a probe that can be located within the magnetic field. Methods of the invention utilize devices that can determine the location of the probe within the magnetic field without the need for a reference probe and a tracked probe within the magnetic field.

One example of a device that can be used as the magnetic field generator and the probe includes a device as described in U.S. Pat. No. 6,288,785 (Frantz et al.), the disclosure of which is incorporated herein by reference.

One embodiment of a method of the invention includes use of a new generation of magnetic field generation based tracking systems, with increased accuracy and the ability to track objects even in ferromagnetic environments. Magnetic tracking systems do not require that a direct line of sight be maintained. In addition, these new magnetic systems use sensors that are extremely small (0.9 mm in diameter and 8 mm in length). This enables the sensors to be placed at the tool tip itself rather than relying on a sensor mounted at the far end of the tool. Tools can also be made of flexible materials, as long as the tool tip containing the sensor remains rigid. These features also make them ideal for percutaneous tracking. The magnetic sensors are small enough to be embedded directly into or next to the anatomical structure to be tracked. Because no line of sight need be maintained, the operating environment remains minimally encumbered.

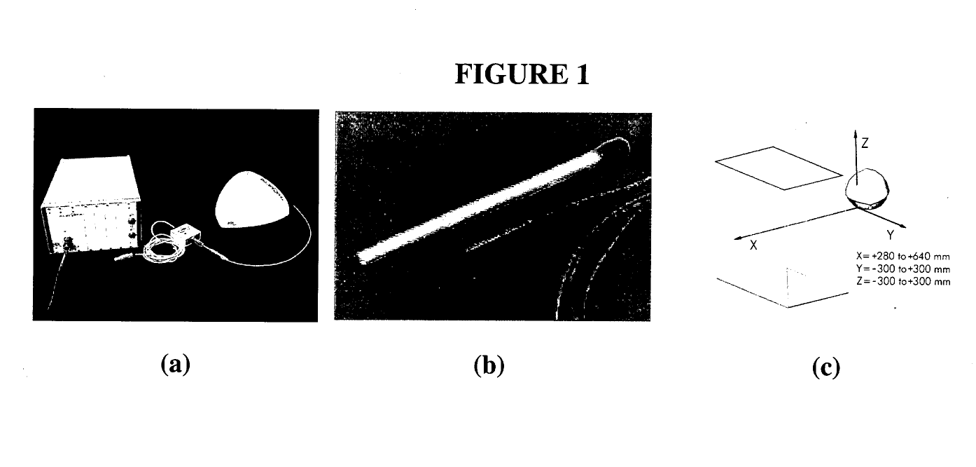

One of these new magnetic tracking systems is the AURORA™ system from Northern Digital Inc., Ontario, Canada. This system is illustrated in FIG. 1. The system consists of a control unit, sensor interface device, sensors, and field generator as shown in FIG. 1a. The sensors (FIG. 1b) plug into the sensor interface unit and can be as small as 0.9 mm in diameter and 8 mm in length. For comparison, the sensor coil is shown next to a match with the leads protruding from the coil. The sensors can have a positional accuracy of 1-2 mm and angular accuracy of 0.5-1 degree. The measurement volume (FIG. 1c) is based on the reference coordinate system of the field generator. The distance along the x-axis is 280 to 640 mm, along the y-axis from −300 to 300 mm, and along the z-axis from −300 to 300 mm. This volume is sufficient to cover the area of interest for abdominal interventions.

In an embodiment of the invention that utilizes the AURORA™ system, the position of the markers can be registered in magnetic space by having the user activate the AURORA™ system and touch each of the markers with the magnetically tracked sensor probe. This functions to locate the markers within the magnetic space, which can then be correlated to the location of the markers within the three-dimensional space.

The step of correlating the magnetic field space with the three-dimensional image space functions to relate the two spaces to each other, which allows a user of the method to see the position of the magnetically tracked probe in the context of the three-dimensional image.

This step can be carried out through mathematically relating the two three-dimensional volumes to each other, using the locations of the imageable, visually apparent markers as points which are known in each space. One example of a method of accomplishing the correlation of the magnetic field space and the three-dimensional space is to utilize a least squares fit. One specific method of accomplishing the least squares regression analysis can be found in S. Umeyama, “Least-squares estimation of two 3-D point sets”, IEEE trans pattern anal. mach. intell., vol. 13, pp. 376-380, 1991.

Overlaying the Magnetic Probe in the Three-Dimensional Image Space

The next step in a method of the invention is to overlay the location of the magnetic probe in the three-dimensional image space. This functions to allow the user to see the location of the magnetic probe, in real-time, in the three-dimensional space that was imaged of the organ or area of interest. This step also functions to allow the user to more easily visualize, in three-dimensions, the location of the magnetically tracked probe.

Once the previous step of correlating the magnetic field space with the three-dimensional space has been accomplished, this step be easily accomplished by displaying the particular portion of the three-dimensional image in which the magnetic probe is currently located.

Planning a Path

The next step in a method of the invention includes planning the path of the magnetic probe within the organ or area of interest. This step functions to allow the user to determine a path to the area within the organ or area of interest that is being targeted. In one embodiment, the area within the organ or area of interest can be a tumor, a specific structure such as an artery or vein, or other anatomy of interest.

In one embodiment, the step of planning a path begins by locating the target within the three-dimensional images. For example, in an embodiment where the three-dimensional image was obtained by a CT scan, the user can scroll through axial images to find a specific image that includes the tumor, for example, and select that as the target. In another embodiment, another step involved in planning the path of the magnetic probe within the organ or area of interest includes selecting a skin entry point. In one embodiment, this can also be accomplished by scrolling through axial images, in the case of utilizing a CT scan as the three-dimensional image.

Selection of the skin entry point and the target define, at least in part, the biopsy path. The biopsy path is a path that is plotted between the skin entry point and the target. The biopsy path can compensate for or consider structures within the organ or area of interest that the user would like to avoid. Alternatively, these areas can be avoided by the choice of skin entry point.

Proceed along Planned Path

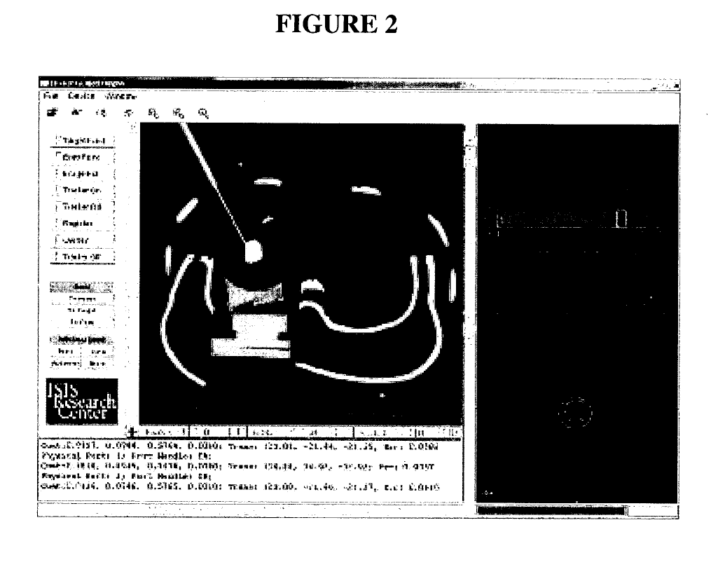

The next step in a method of the invention is for the user to proceed along the planned path. In one embodiment, the user is aided in this step, as well as others, by the use of a graphical interface. FIG. 2 depicts one embodiment of the user interface. The user interface can include a procedure bar 110, a main window 100 showing the three-dimensional image and an overlay of the probe, and a targeting window 120. Alternatively, the user interface can include a respiratory monitor.

The procedure bar 110 allows the user to control certain aspects of the device through a computer. For example, in one embodiment, the user can modify the display of the user interface itself, designate a specific point of the magnetic probe as the skin entry point, turn the magnetic tracking on or off, register the imageable, and visible markers within the magnetic space. Other embodiments can have more, different, or less aspects to control.

The main window 100 shows the three-dimensional image with an overlay.

This window functions to display the correlated three-dimensional image and magnetic field space. In one embodiment, this display provides a simultaneous view of the anatomy, as captured by the three-dimensional imaging technique, and a view of the magnetic probe. The display in this window can be updated to monitor the location of the probe. In one embodiment, as the prove is moved across the area of the three-dimensional image, different axial or oblique images will be displayed indicating the three-dimensional image that corresponds with the location of the magnetically tracked probe.

The targeting window 120 provides the user with assistance in proceeding along the planned path. In one embodiment, the targeting window provides three separate indications.

A first indicator shows the proximity of the magnetic probe tip to the chosen skin entry point. In the embodiment shown in FIG. 2, the proximity is shown by the location of the small circle with respect to the crosshairs. It should of course be understood that this relationship could also be shown in other ways, such as for example, distance from the skin entry point.

A second indicator shows the position of the opposite end of the magnetic probe in relation to the planned path. This indicator functions to inform the user whether the trajectory of the magnetic probe is in line with the planned path. This indicator is shown by the location of the larger circle with respect to the crosshairs, but could again be shown in other ways.

Once the user accurately places the tip of the magnetic probe on the skin entry point, as shown by the first indicator and positions the opposite end of the magnetic probe in line with the planned path as is shown by the second indicator, the path of the magnetic probe from the skin entry point to the target will be along the planned path (within any error caused by having the skin entry point or the trajectory of the needle not perfectly lined up).

A third indicator shows the depth of the magnetic probe in relation to the depth of the target. This indicator functions to show the user how far the magnetic probe has to be advanced along the pathway to “hit” the target. In one embodiment, this indicator is shown by the progress bar on the bottom of the targeting window 120. In one embodiment, this progress bar fills in as the tip of the magnetic probe gets closer to the target. In another embodiment, the progress bar can both fill up and change colors as the tip of the magnetic probe gets closer to the target.

The graphical user interface can be accomplished through the use of any programming software that allows a skilled user to set up and develop a graphical user interface for the specific application desired. One example of such a software program includes FLTK. FLTK is a cross-platform C++ GUI toolkit for UNIX®/Linux® (X11), Microsoft® Windows®, and MacOS® X. The FLTK software can be obtained via the FLTK website with the address www.fltk.org.

In one embodiment, the step of proceeding along the proposed path can be accomplished by locating the skin entry point by using a first indicator, locating the trajectory of the magnetic probe by using a second indicator, and advancing the magnetic probe to the target by inserting the magnetic probe along the planned path until the progress meter indicates that the target has been “hit”.

In one embodiment of the invention, the step of proceeding along the planned path includes magnetically tracking the magnetic probe. This step functions to continuously monitor the location of the magnetically tracked probe in the magnetic field. The location within the magnetic field is correlated to the three-dimensional image, through use of the graphical interface to aid the user in placing and inserting the magnetically tracked probe.

In one embodiment of the invention, the AURORA™ system, as discussed above is used to track the magnetically tracked probe. One of skill in the art, having read the instant specification would understand that other magnetic tracking systems can also be used. It should also be understood that other non-line of sight tracking systems could also be utilized in methods of the invention. The magnetically tracked probe can be incorporated into various medically relevant instruments. For example, the magnetically tracked probe can be incorporated into a needle, a catheter, a camera, a source of radiation, or other surgical instruments.

The magnetically tracked probe can then be used to direct the user within the organ or area of interest. Such a method can be useful for a number of different applications. For example, RF tumor ablation, liver biopsy, transjugular intrahepatic portosystemic shunt (TIPS), and the like can all be accomplished using the methods of the invention.

Another embodiment of a method of the invention begins by acquiring a three-dimensional image of the organ or area of the subject in interest. The three-dimensional image is acquired with at least three imageable, visible markers and at least one magnetically tracked marker in place. After the image has been acquired, a magnetic field is generated in an area of the organ or area of the subject. Then, the position of the imageable, visible markers and the magnetically tracked marker in the generated magnetic field is recorded. Once the position of the imageable, visible markers and the magnetically tracked markers are located, the three-dimensional image space is correlated with the magnetic field space. Next, a probe is introduced into the area of the organ or area of interest in the subject. As the probe is moved in the organ or area of interest, the position of the probe is tracked in the generated magnetic field. The method allows three-dimensional imaging by correlating the position of the probe in the generated magnetic field with the position of the surface markers and the magnetic marker in the generated magnetic field and the three-dimensional image.

One example of a clinical scenario for using this system to demonstrate percutaneous abdominal interventions begins by wedging a magnetically tracked catheter in the hepatic vein of the liver. Several skin fiducials are also placed on the rib cage. Next, a liver phantom simulator is placed in a CT scanner. A series of thin 1-2 mm axial slices are obtained from the base of the lungs through the liver while the liver is kept in end inspiration (simulating the breath-hold technique used in clinical practice). The catheter is left in place and the simulator is moved to the interventional table. A magnetic field generator is placed near the liver, and the position of the catheter is then read in magnetic space. The position of the skin fiducials are also read in magnetic space by touching each fiducial with a magnetically tracked probe. Using the locations determined above, the position of the catheter and fiducials is determined in CT space by asking the user, for example, an interventional radiologist to select these points on the CT images.

A least-squares fit registration algorithm is then utilized to determine the transformation matrix from magnetic space to CT space. The interventionalist uses the magnetic probe to approach the liver as he/she would during percutaneous liver biopsy or tumor ablation. The probe is tracked in real-time and the transformation matrix computed above is used to compute the overlay of the probe on the CT images.

A monitor is utilized to display cross sectional CT images of the liver which are reformatted in an off-axial plane parallel to the magnetic probe. This allows the interventionalist to view the projected path of the instrument in real-time. The cross sectional image can be displayed either with the motion platform stopped (simulating a breath hold) or while the liver is moving (simulating a respiring patient). If the liver is moving, the magnetically tracked catheter is used to update the current position of the liver.

### Working Examples

The following examples provide an illustration of the advantages of certain embodiments of the invention.

### EXAMPLE 1

This example illustrates one specific configuration of a device that can carry out the method of the invention.

To evaluate magnetic tracking for minimally invasive abdominal interventions, a liver respiratory motion simulator was developed. The simulator includes a synthetic liver mounted on a motion platform. The simulator consists of a dummy torso, a synthetic liver model, a motion platform, a graphical user interface, the AURORA™ magnetic tracking system, and a magnetically tracked needle and catheter as described herein.

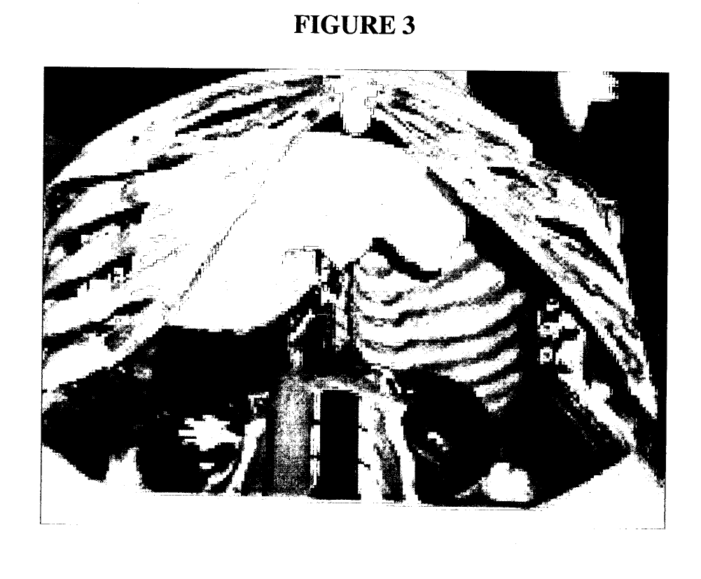

A human torso model containing a liver phantom was made from a two part flexible foam (FlexFoam III, Smooth-On, Easton Pa.) which was cast from a custom made mold. The foam material was cured to approximately simulate the resistance of the liver tissue to needle puncture. Two spiculated, silicone, elliptical tumors (maximum diameters of 3.1 and 2.2 cm) containing radio-opaque CT contrast were incorporated into the liver model prior to curing to serve as tumor targets. The liver was attached to a linear motion platform at the base of the torso's right abdomen. A depiction of the human torso model with the liver phantom attached is seen in FIG. 3.

The platform can be programmed to simulate the physiological cranio-caudal motion of the liver with options for respiratory rate control, breath depth, and breath pause (to simulate a clinically utilized breath hold). A ribcage and single layer latex skin material (Limbs and Things, Bristol, UK) were added for aesthetic and physical reality.

A magnetic field based tracking system, the AURORA™ (Northern Digital Inc., Waterloo Ontario, Canada), was used in the experiments. The system consists of a control unit, sensor interface device, and field generator as shown in FIG. 1a.

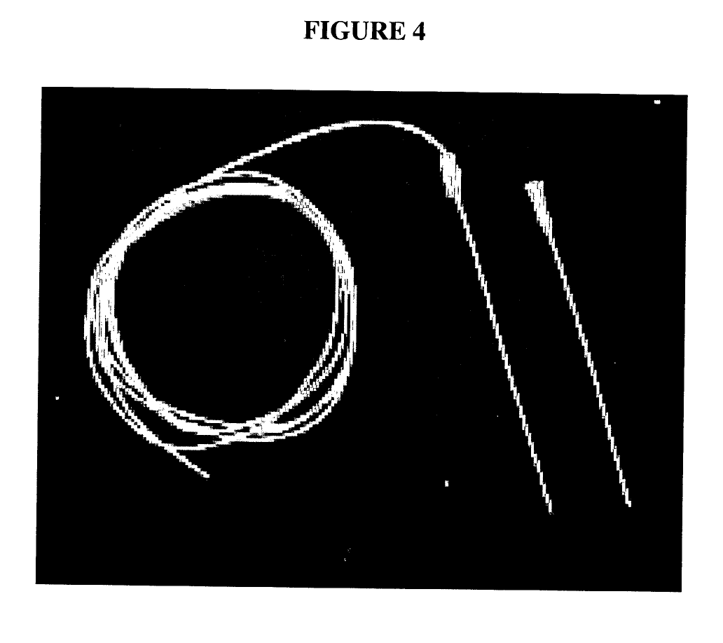

The AURORA™ uses cylindrically shaped sensors that are extremely small (0.9 mm in diameter and 8 mm in length). This enables the sensors to be embedded into surgical instruments. Two magnetically tracked surgical instruments were used in this experiment: 1) a 5-French catheter with an embedded sensor coil (Northern Digital Inc.); and 2) a MagTrax needle/probe combination (Traxtal Technologies, Houston, Tex.) as shown in FIG. 4. The MagTrax needle/probe includes a 15 cm stylette with a magnetic sensor at its tip and an 18-gauge trocar. This magnetically tracked instrument was used to puncture the tumors in Example 3.

A PC-based software application was developed to assist the user in performing the puncture of the liver parenchyma and needle guidance into the liver tumors. The system incorporates a graphical user interface (FIG. 2). The user interface allowed the serial axial CT images to be loaded into the system, the creation of a pre-procedural plan to the target of interest, tracking of respiratory motion, and real-time display of the magnetically tracked instrument as it moves in magnetic space, for example, as it approaches the target tumor.

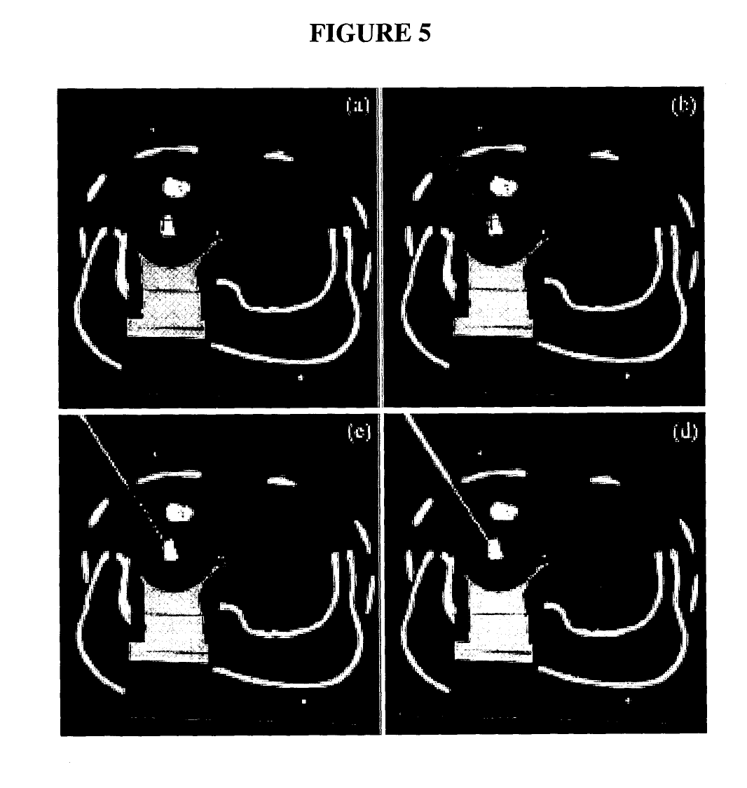

The sequence of steps in path planning and needle placement is shown in FIG. 5 and detailed in Example 3. First, the target tumor is selected by the user on an axial image of the phantom torso. Next, the user selects the skin entry point (FIG. 5a), and a planned path appears on the reconstructed three-dimensional image (FIG. 5c). The needle/probe is then placed at the skin entry point using the cross hairs targeting window (FIG. 5b). Last, the needle/probe is driven into the tumor along the planned path indicated by the dotted line in FIG. 5c (FIG. 5d) to the depth of the targeted tumor.

### EXAMPLE 2

To test the system described in Example 1, a simulated transjugular intrahepatic portosystemic shunt (TIPS) procedure was carried out using the foam liver phantom and the respiratory motion simulator describe in Example 1. A foam liver was cast with two barium coated straws and mounted to the one degree of freedom motion platform. A rib cage was taken from an anatomical model and placed over the moving liver. Fiducials were mounted on the rib cage (multi-modality radiographic markers, IZI Medical, Baltimore, Md.).

A special catheter, containing a magnetically tracked sensor coil, was inserted into the liver simulating the insertion of a coaxial catheter into the hepatic vein during the TIPS procedure. A pre-procedure CT scan was done (5 mm collimation with 1 mm reconstruction, 219 slices total). The scan was transferred to the user interface using the DICOM (Digital Imaging and Communications in Medicine) protocol.

The desired path was then planned thorough the use of the user interface by the user by selecting the skin entry point and the at least one target point. The magnetic tracking system was then used to track the probe and provide image guidance as described above.

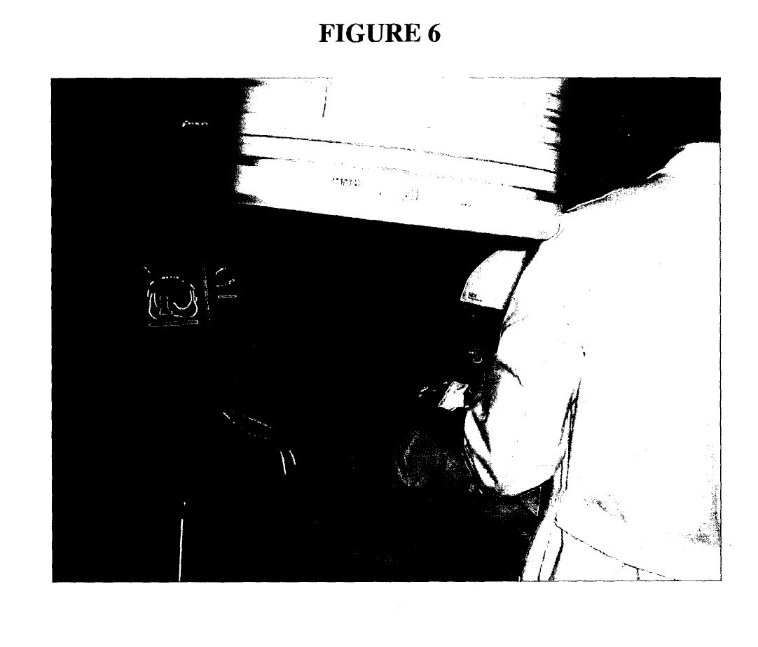

Using the targeting window, the probe (actually a magnetic tracked needle) was placed on the skin entry point and then aligned along the desired trajectory. The targeting window consists of circles representing the tip and handle of the needle along with crosshairs indicating the target point. This interface was adopted as it felt that aligning the circles was easier than a direct anatomical view, particularly if the liver is moving. The needle was driven into the liver along this planned trajectory until the desired depth was indicated. The actual position of the needle was then confirmed by fluoroscopy as shown in FIG. 6. Both “vessels” were successfully punctured with a single needle pass as can be seen in these images. This puncture would replace the difficult portosystemic venous puncture needed during a typical TIPS procedure.

### EXAMPLE 3

A series of tumor targeting experiments were performed to test the accuracy of the system of Example 1 above in guiding a user to a target while the phantom liver resumes physiologic respiration. Two users independently performed 8 punctures each according to the following method.

Stage 1: CT scanning and registration

A magnetically tracked catheter was wedged into the hepatic vein of the phantom liver. Several skin fiducials (multimodality markers, IZI Medical, Baltimore, Md.) were placed on the rib cage.

A series of 3 mm axial slices with 1 mm axial reconstructions were obtained on CT VolumeZoom (Siemens, Erlangen, Germany) from the base of the lungs through the liver while the liver was kept in end-inspiration (simulating the breath-hold technique used in clinical practice).

The images were transferred to the graphical user interface using the DICOM standard.

The tracking catheter was left in the hepatic vein and the simulator was moved to the interventional radiology suite. The magnetic field generator was positioned near the phantom above the chest.

The position of the wedged catheter was read in the magnetic coordinate system. The position of the skin fiducials were read in the magnetic coordinate system by touching each fiducial with the MagTrax needle.

The position of the catheter and fiducials was determined in CT coordinate space by prompting the user to select these same points on the CT images.

A least-squares fit registration algorithm was invoked to determine the transformation matrix from magnetic space to CT space.

Stage 2: Biopsy path planning

Each user was allowed one practice “planning phase” and “puncture (biopsy) phase” to become familiarized with the user interface.

The user selected the target and a suitable skin entry point by scrolling through the axial images thus selecting a biopsy path.

Simulated respirations were initiated at 12 breaths per minute with 2 cm cranio-caudal liver excursion.

Stage 3: Biopsy

The MagTrax needle/probe was positioned on the skin entry point as determined in the “planning phase” and displayed by the overlay in the graphical user interface.

A real-time display of the current liver position was displayed by the graphical user interface system based on the position of the magnetically tracked catheter.

The MagTrax needle was tracked in real-time and the transformation matrix computed above was used to compute the overlay of the probe on the CT images which were reconstructed to show the planned path of the needle.

When the user was satisfied with the targeted position relative to the planned path, the user would initiate temporary cessation of respiration (simulating a 20 second breath hold in clinical practice). If the allotted time was exceeded, the phantom would continue spontaneous respirations for a minimum of 20 seconds (hyperventilation in clinical practice). Any partially inserted needle would be left in place as is frequently done during biopsy procedures.

Repeating the above step, the user would keep making minor adjustments to the needle until satisfied with the needle position as displayed on the graphical user interface.

The time for each “planning phase” and “biopsy phase” were recorded. Multi-projection fluoroscopic images were taken at the end of each needle placement to ascertain whether the target tumor was successfully punctured.

An optical passive tracking system was used to compare the performance of the magnetically tracked system. The MagTrax needle/probe containing the single five degree of freedom magnetically tracked sensor solidly fixed to two passive optically tracked rigid bodies ( small 50×50 mm and large 95×95 mm). The sensor assembly was moved randomly through 101 positions in a volume of 36 mm×26 mm×47 mm. At each location the sensor assembly was clamped and 10 samples from each of the targets were collected by the POLARIS ® ( optical system (Northern Digital Inc., Ontario Canada) and AURORA™ magnetic system (Northern Digital Inc., Ontario Canada). The data sets were aligned by mathematical transformations and the difference in position and orientation of the two POLARIS® sensors (control) versus the larger POLARIS® sensor and MagTrax probe were calculated over the 101 positions. This experiment was performed in the absence of ferromagnetic interference.

The mean measurement error and standard deviation of the MagTrax needle/probe using the AURORA™ system was 0.71+0.43 mm (n=101) in a non-surgical environment. The maximum error noted was 2.96 mm.

The targeted tumor was successfully punctured in 14 out of 16 biopsy attempts (87.5%). This was done without any additional real-time imaging guidance such as fluoroscopy. Instead, fluoroscopy was used to confirm the final location of the needle and evaluate the accuracy of the system.

Each user missed the target tumor once. In those instances, the maximal tangential distance from the lesion to the needle was 3.98 mm. On most occasions, the user was able to reach the tumor in a single continuous puncture after the needle was positioned on the skin entry point. This was done within a single 20 second breath hold (pause in liver motion) in end-inspiratory liver position. More than two breath hold cycles with intervening period of hyperventilation were needed on only 1 out of 16 experimental trials. The time needed for registration ranged from 173 to 254 seconds. The planning time, needle manipulation time, and total procedure times for the 16 trials are presented in Table 1 below.

TABLE 1

| | Mean Planning | Needle Manipulation | Total Procedure |
|---|---|---|---|
| | Time (s) ± SD | Biopsy Time (s) ± SD | Time (s) ± SD |
| User 1 | 72 ± 35 | 79 ± 40 | 151 ± 59 |
| User 2 | 61 ± 31 | 111 ± 41 | 172 ± 43 |
| Overall | 71 ± 36 | 93 ± 43 | 163 ± 57 |

The results presented here show the feasibility of magnetic tracking in combination with pre-planning of a path and computer guided use of the magnetic tracking system. The accuracy of the MagTrax needle/probe used with the AURORA™ was measured as 0.71 mm. Additionally, the location of the magnetic sensor in the tip of the needle/probe means the instrument is not subject to errors introduced by needle bending unlike some systems of the prior art where the proximal end of the needle is tracked.

The graphical user interface utilized herein allowed a high success rate (87.5%) for needle punctures of the two small to medium sized simulated tumors. Most notably, the procedure was done while actively tracking the physiological motion of the liver. The system was easy to use requiring only a single practice attempt to attain a satisfactory comfort level with the system. The entire average procedure time lasted less than three minutes which is shorter than the time needed to perform the task during a conventional CT guided biopsy.

### EXAMPLE 4

The device of Example 1 was utilized to test simultaneous needle puncture of two vessels in a phantom liver.

An abdominal torso phantom (Anatomical Chart Co., Skokie, Ill.) was modified by removing the ventral abdominal wall and placing a servomotor-driven platform mount in the “paraspinal” area upon which a foam liver phantom was secured. The liver phantom contains target thin-walled “vascular structures” created by the removal of barium-coated plastic drinking straws placed within the foam mixture prior to final casting. The resulting air-filled tubes measure approximately 5 mm in diameter. The phantom was moderately more firm than the human liver with respect to the tactile sense during needle puncture. The servomotor control system produces linear platform motion which simulates the respiratory motion of the liver.

The device described in Example 1 was used with the following modifications. The catheter-based fiducial was placed through the simulated intrahepatic inferior vena cava and into a simulated hepatic vein and fixed in position with a small amount of adhesive. All motion of the liver phantom was therefore tracked by the embedded catheter-based fiducial. The tracked puncture needle was a modified 18 gauge trocar needle with the coil fiducial placed in the stylet (Traxtal Technologies, Bellaire, Tex.).

For each series of puncture experiments, a total of four skin fiducials were placed on the anterior costal margins. The phantom was placed in a Siemens CT scanner and contiguous 1 mm images of the liver were obtained. The CT DICOM dataset was transferred to a Windows® NT workstation where the axial images were displayed and reviewed in a single window on the graphical user interface.

The target vessels were selected and a linear puncture needle trajectory was highlighted. The magnetic field generator was placed next to the torso. The registration process was done using the external and catheter-based fiducials. The skin fiducials were identified on the CT images and automatic segmentation was performed to identify the isocenter of each fiducial. The tracked needle was then placed on each fiducial sequentially, thereby recording the position in magnetic space. The catheter-based fiducial was registered in the end-expiratory phase position by identifying the tip of the catheter containing the coil fiducial on the respective CT image. In all experiments, the registration error (root mean square) was 1-2 mm.

The skin entry site was determined by placing the tracked needle on the “skin” of the torso, guided in real-time in a third window which displayed the position of the needle tip relative to the previously determined needle trajectory. The correct needle “depth” was compared to the termination target position, and needle advancement ceased when the system graphically indicated the desired needle depth.

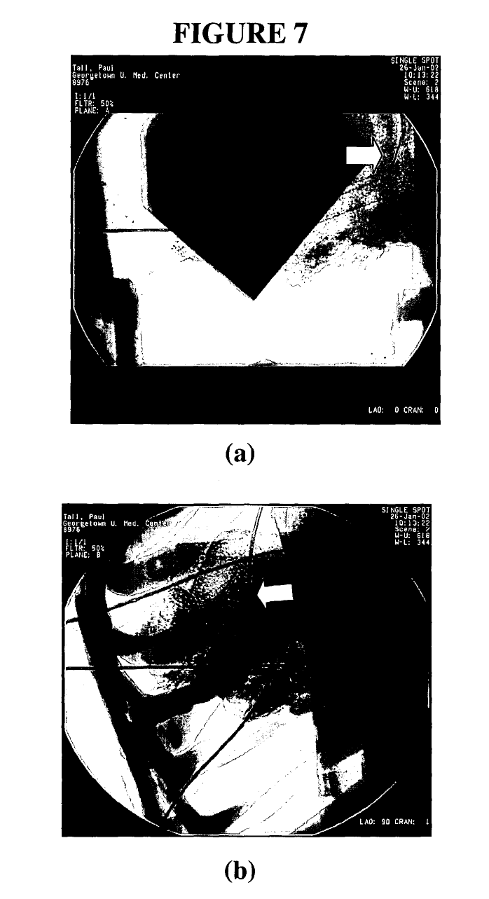

In initial tests, simultaneous needle puncture of two vessels was performed in the stationary liver phantom to simulate the key step in the specifically modified TIPS procedure. Needle placement was performed by hand by experienced and less experienced operators. Orthogonal biplane fluoroscopic images of the liver phantom were then obtained which confirmed successful puncture of both targets by the single needle pass (FIG. 7).

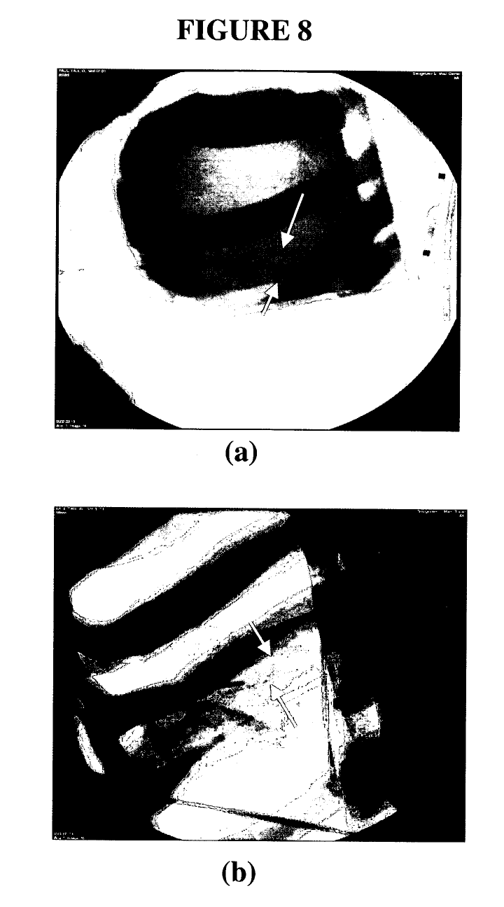

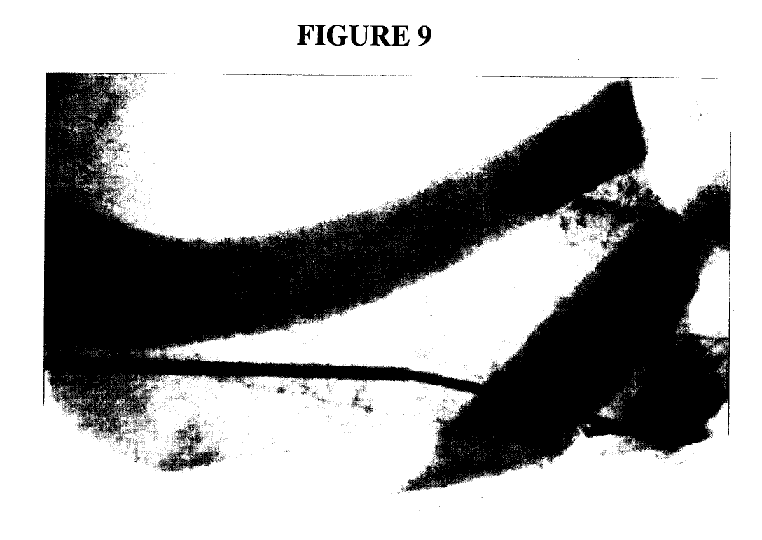

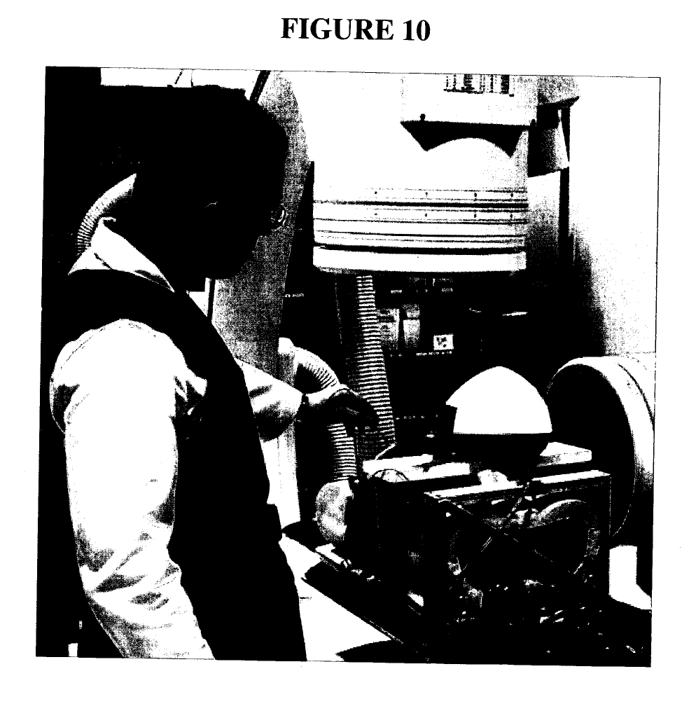

In a second liver phantom, a single vessel served as a target, and guided needle punctures were performed by a single operator on ten occasions during simulated respiratory motion. The respiratory motion ranged from a frequency of 12 to 40/minute and an excursion distance of 1 to 2 cm. Orthogonal biplane digital images were obtained for each needle pass to confirm successful target puncture (FIG. 8). A “guidewire test” was then performed consisting of an attempt to pass a standard angiographic 0.035 inch guidewire through the needle into the targeted “vessel” (FIG. 9). The time required to successfully puncture the vessel target after placing the needle tip on the skin was recorded for each needle pass. A picture of the interventional suite and experimental set-up is shown in FIG. 10.

For needle passes performed during respiratory excursions, success was defined as 1) determination of the needle tip position within the vessel lumen by orthogonal digital images, and 2) successful passage of the guidewire without needle manipulation. For the 10 attempted passes, 8 passes were completely successful. In the remaining two passes, orthogonal biplane images demonstrated the needle tip within the target vessel but in an eccentric position, although withdrawal of the needle tip by 1 mm or rotation of the needle was required to allow successful passage of the guidewire. Needle puncture attempts averaged 28.6 sec (standard deviation 34.1 sec), with a prolonged attempt lasting 105 seconds caused by significant needle deflection within the phantom attributed to incorrect insertion of the stylet within the trocar. Needle misalignment was immediately recognized in this case, and needle redirection resulted in a successful puncture.

In all instances, the graphical user interface provided a user-friendly, concise, and stepwise program for needle trajectory planning and needle placement. The rapid needle position update rate provided by the tracking system and interface allows for the real-time display of the position of the needle alignment and depth parameters. The intravascular, fixed catheter-based fiducial permits direct tracking of the respiratory related organ motion for real-time needle placement.

The above specification, examples and data provide a complete description of the manufacture and use of the composition of the invention. Since many embodiments of the invention can be made without departing from the spirit and scope of the invention, the invention resides in the claims hereinafter appended.

The claimed invention is:

1. A method of providing image guidance for use in an organ or area of interest subjected to motion, said method comprising the steps of:
a. acquiring a three-dimensional image of the organ or area of interest of the subject with at least two imageable, visible markers and at least one magnetically tracked marker in place;
b. acquiring a three-dimensional image of the organ or area of the subject with at least two imageable, visible markers and at least one magnetically tracked marker in place;
c. correlating a magnetic field space to the three-dimensional image space;
d. providing an overlay of a magnetically tracked probe in the three-dimensional image space;
e. planning a path to a target within the organ or area of interest within the subject; and
f. proceeding along the planned path, wherein the probe is tracked along the planned path.

2. The method of claim 1, wherein the three-dimensional image is a CT image.

3. The method of claim 1, wherein the step of correlating comprises making contact between the at least two imageable, visible markers and the magnetically tracked probe.

4. The method of claim 1, wherein the step of correlating comprises a least squares regression analysis.

5. The method of claim 1, wherein the step of planning a path to the area within the organ or area of interest comprises determining a skin entry point.

6. The method of claim 1, wherein the step of planning a path to the area within the organ or area of interest comprises determining a target within the organ or area of interest.

7. The method of claim 1, wherein the step of proceeding along the planned path comprises use of a magnetic tracking system.

8. The method of claim 7, wherein the magnetic tracking system comprises an AURORAT™ system.

9. The method of claim 1, wherein the step of proceeding along the planned path comprises the use of a graphical interface.

10. The method of claim 9, wherein the graphical interface comprises:
a.) a main window showing the overlay of the magnetic probe and the three-dimensional image; and
b.) a targeting window which provides assistance to proceed along said planned path.

11. The method of claim 10, wherein the targeting window comprises a first indicator and a second indicator.

12. The method of claim 11, wherein the first indictor provides proximity of the tip of the magnetically tracked probe to the planned skin entry point.

13. The method of claim 11, wherein the second indicator provides proximity of the hub of the magnetically tracked probe to the planned skin entry point.

14. The method of claim 11, wherein the targeting window further comprises a depth indicator.

15. The method of claim 14, wherein the depth indicator shows the depth of the tip of the magnetically tracked probe in relation to the depth of the target.

16. A method of providing image guidance for use in an organ or area of interest subjected to motion, said method comprising the steps of:
a. acquiring a three-dimensional image of the organ or area of the subject in interest with at least three imageable, visible markers and at least one magnetically tracked marker in place;
b. generating a magnetic field in an area of the organ or area of interest;
c. recording the position of the imageable, visible markers and the magnetically tracked marker in the generated magnetic field;
d. correlating the three-dimensional image space with the magnetic field space;
e. introducing a magnetically tracked probe into the area of the organ or area of interest;
f. tracking the probe as it moves in the organ or area of interest;
g. planning a path to a target; and
h. proceeding along the planned path to the target through use of a graphical user interface.

17. A method of providing image guidance for use in an organ or area of interest subjected to motion, said method comprising the steps of:
a. acquiring a CT image of the organ or area of the subject in interest with at least three imageable, visible markers and at least one magnetically tracked marker in place;
b. generating a magnetic field in an area of the organ or area of interest;
c. recording the position of the imageable, visible markers and the magnetically tracked marker in the generated magnetic field, wherein the position of the imageable, visible markers are recorded through use of a magnetically tracked probe;
d. correlating the three-dimensional image space with the magnetic field space;
e. introducing a magnetically tracked probe into the area of the organ or area of interest;
f. overlying an image of the CT scan with the position of the magnetically tracked probe;
g. tracking the probe as it moves in the organ or area of interest, whereby the position of the magnetically tracked probe with respect to the image of the CT scan is updated in real-time;
h. planning a path to a target by using identifying a skin entry point and a target; and
i. proceeding along the planned path to the target through use of a graphical user interface which indicates at least the proximity of the magnetically tracked probe to the skin entry point.

18. The method of claim 17, wherein the graphical user interface further indicates the proximity of the trajectory of the magnetically tracked probe to the planned path.

19. The method of claim 17, wherein the graphical user interface further indicates the depth of the magnetically tracked probe with respect to the depth of the target.
